(19) United States
(12) Patent Application Publication
(10) Pub. No.: US 2007/0048745 A1
(43) Pub. Date: Mar. 1, 2007

(54) Systems and methods for partitioned nanopore analysis of polymers

(76) Inventors: Timothy Herbert Joyce, Mountain View, CA (US); Jerry T. Dowell, Carson City, NV (US)

Correspondence Address: AGILENT TECHNOLOGIES INC., INTELLECTUAL PROPERTY ADMINISTRATION, M/S DU404, P.O. BOX 7599, LOVELAND, CO, 80537-0599, US

(21) Appl. No.: 11/214,546

(22) Filed: Aug. 30, 2005

(51) Int. Cl. C12Q 1/68; C12M 1/34

(52) U.S. Cl. 435/6; 435/287.2; 977/924

(57) ABSTRACT

Devices, systems, and methods for nanopore analysis of polymers are provided. One exemplary device, among others, includes a substrate having a plurality of partitioned nanopores configured to receive a polymer sample. In addition, the device includes a plurality of sets of resonant tunneling electrodes adjacent the partitioned nanopore. At least one set of resonant tunneling electrodes is configured to detect tunneling current as monomers of a polymer in the polymer sample sequentially travel through at least one partitioned nanopore.

### BACKGROUND

Determining the nucleotide sequence of DNA and RNA in a rapid manner is a major goal of researchers in biotechnology, especially for projects seeking to obtain the sequence of entire genomes of organisms. In addition, rapidly determining the sequence of a nucleic acid molecule is important for identifying genetic mutations and polymorphisms in individuals and populations of individuals.

Nanopore sequencing is one method of rapidly determining the sequence of nucleic acid molecules. Nanopore sequencing is based on the property of physically sensing the individual nucleotides (or physical changes in the environment of the nucleotides (i.e., electric current)) within an individual polynucleotide (e.g., DNA and RNA) as it traverses through a nanopore. In principle, the sequence of a polynucleotide can be determined from a single molecule. However, in practice, it is preferred that a polynucleotide sequence be determined from a statistical analysis of data obtained from multiple passages of the same molecule or the passage of multiple molecules having the same polynucleotide sequence. The use of membrane channels to characterize polynucleotides as the molecules pass through the small ion channels has been studied by Kasianowicz et al. (Proc. Natl. Acad. Sci. USA. 93:13770-3, 1996, incorporate herein by reference) by using an electric field to force single stranded RNA and DNA molecules through a 2.6 nanometer diameter nanopore (i.e., ion channel) in a lipid bilayer membrane. The diameter of the nanopore permitted only a single strand of a polynucleotide to traverse the nanopore at any given time. As the polynucleotide traversed the nanopore, the polynucleotide partially blocked the nanopore, resulting in a transient decrease of ionic current. Since the length of the decrease in current is directly proportional to the length of the polynucleotide, Kasianowicz et al. were able to experimentally determine lengths of polynucleotides by measuring changes in the ionic current.

Baldarelli et al. (U.S. Pat. No. 6,015,714) and Church et al. (U.S. Pat. No. 5,795,782) describe the use of nanopores to characterize polynucleotides including DNA and RNA molecules on a monomer by monomer basis. In particular, Baldarelli et al. characterized and sequenced the polynucleotides by passing a polynucleotide through the nanopore. The nanopore is imbedded in a structure or an interface, which separates two media. As the polynucleotide passes through the nanopore, the polynucleotide alters an ionic current by blocking the nanopore. As the individual nucleotides pass through the nanopore, each base/nucleotide alters the ionic current in a manner which allows the identification of the nucleotide transiently blocking the nanopore, thereby allowing one to characterize the nucleotide composition of the polynucleotide and perhaps determine the nucleotide sequence of the polynucleotide.

One disadvantage of previous nanopore analysis techniques is the inability to analyze a large volume of target polymers in one run. Moreover, existing nanopore techniques do not provide for multiple sequencing of a single species of polymer present in a heterogeneous sample.

U.S. Patent Application Publication Nos. 20040149580 and 20040144658 to Flory disclose the use of resonant tunneling electrodes to sequence biopolymers. Because the location of a biopolymer with regard to set of resonant tunneling electrodes can significantly affect tunneling current values, the degree of alignment of the biopolymers as they are being detected determines the level of accuracy of the sequencing method. Accordingly, there is an need for methods and systems that increase the alignment of analytes for characterization by resonant tunneling electrodes.

### SUMMARY

Devices, systems and methods for nanopore analysis of polymers are provided. One exemplary device, among others, includes a substrate having a plurality of partitioned nanopores configured to receive a polymer sample. In addition, the device includes a plurality of sets of resonant tunneling electrodes adjacent the partitioned nanopore. At least one set of resonant tunneling electrodes is configured to detect tunneling current as monomers of a polymer in the polymer sample sequentially travel through at least one partitioned nanopore.

Another exemplary device, among others, includes a plurality of nanopores disposed on a substrate for receiving fractions of a polymer sample, a partitioning grid operatively coupled to the plurality of nanopores for segregating each of the plurality of nanopores, and a plurality of resonant tunneling electrodes configured to detect tunneling current as monomers of a polymer in the polymer sample sequentially travel through each of the plurality of partitioned nanopores.

An exemplary nanopore analysis system for determining the sequence of a target polynucleotide, among others, includes: a plurality of capillary electrophoresis devices, each of the plurality of capillary electrophoresis devices independently and operatively coupled to a partitioned nanopore, and a resonant tunneling electrode independently and operatively coupled to the partitioned nanopore. The resonant tunneling electrode is configured to detect tunneling current through a polymer as monomers of the polymer sequentially travel through the partitioned nanopore.

An exemplary method for characterizing an analyte, among others, includes: receiving the analyte through a partitioned nanopore, and detecting tunneling current from the analyte with a set of resonant tunneling electrodes disposed adjacent the partitioned nanopore.

An exemplary method for sequencing a polynucleotide, among others, includes: receiving an amplified polynucleotide sample into a capillary operably coupled to a plurality of partitioned nanopores positioned in predetermined locations; providing an electric field across the capillary to electrophoretically separate the amplified polynucleotide sample into fractions, wherein each fraction comprises at least two polynucleotides having about the same number of monomers; determining the sequence of each of the two polynucleotides in at least one fraction by detecting tunneling current through the two polynucleotides with a resonant tunneling electrode as the two polynucleotides individually travel through at least one partitioned nanopore in fluid communication with the capillary; and determining a statistically significant sequence of the amplified polynucleotide based on the detected tunneling currents by correlating the detected tunneling currents to predetermined tunneling currents indicative of specific monomers.

An exemplary method for simultaneously determining the sequence of more than one target polynucleotide, among others, includes: separating a mixture of polynucleotides having different nucleic acid sequences into separate groups, wherein each group comprises polynucleotides of the same sequence; simultaneously receiving each group of polynucleotides into a separate partitioned nanopore; simultaneously detecting the tunneling current from the each polynucleotide within each separate group with a set of resonant tunneling electrodes disposed adjacent each partitioned nanopore; and determining a statistically significant sequence of each group of polynucleotides based on the detected tunneling currents.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Reference is now made to the following drawings. Note that the components in the drawings are not necessarily to scale.

FIG. 1 shows a schematic of an exemplary embodiment of a nanopore analysis system.

FIG. 2 shows a diagram of a representative electrophoretic device that can be used in the nanopore analysis system of FIG. 1.

FIG. 2a shows a diagram illustrating the separation of polymers in an exemplary electrophoretic device.

FIG. 2b shows a diagram of an alternative embodiment of an electrophoretic device in combination with a plurality of nanopore devices.

FIG. 3 shows a diagram of a representative nanopore device.

FIG. 4 shows a diagram of another embodiment of the partitioned nanopores.

FIG. 4a shows a diagram of an alternative embodiment of partitioned nanopores.

FIG. 4b shows a diagram of an exemplary partitioned nanopore.

FIG. 4c shows a diagram of a plurality of exemplary partitioned nanopores.

FIG. 5 shows a flow diagram of an exemplary method according to the present disclosure.

### DETAILED DESCRIPTION

Definitions

The term “nanopore” refers to an opening of about 100 nm or less at its widest point. The aperture can be of any geometric shape or configuration, including, but not limited to, square, oval, circular, diamond, rectangular, star, or the like.

The term “polymer” refers to a composition having two or more units or monomers attached, bonded, or physically associated to each other. The term polymer includes biopolymers.

A “biopolymer” refers to a polymer of one or more types of repeating units. Biopolymers are typically found in biological systems and particularly include polysaccharides (such as carbohydrates), peptides (which term is used to include polypeptides and proteins), glycans, proteoglycans, lipids, sphingolipids, known biologicals materials such as antibodies, etc., and polynucleotides, as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in hydrogen bonding interactions, such as Watson-Crick type, Wobble type and the like. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups, one or both of which may have removable protecting groups).

“Electrophoresis” refers to the motion of a charged particle or polymer, for example a colloidal particle, under the influence of an electric field.

“Entangled polymer solutions” refers to solutions in which polymers can interpenetrate each other. This causes entanglements and restricts the motion (reptation) of the molecules to movement along a ‘virtual tube’ that surrounds each molecule and is defined by the entanglements with its neighbors.

The term “gel” refers to a network of either entangled or cross-linked polymers swollen by solvent. The term is also used to describe an aggregated system of colloidal particles that forms a continuous network.

“Statistically significant” refers to a result that is unlikely to have occurred randomly. “Significant” means probably true (not due to chance). Generally, a statistically significant sequence means a sequence that has about a 5% or less probability of including a random sequence error.

The term “partitioned nanopore” refers to a nanopore surrounded by a barrier which physically separates the nanopore from other nanopores. Generally, the barrier is not in the same plane as the nanopore. The barrier is typically perpendicular to the substrate containing the nanopore. An exemplary barrier is a circular barrier surrounding the perimeter of the nanopore, but it will be appreciated that the barrier can be of any geometric shape so long as it physically separates the nanopore from other nanopores in the same plane.

Exemplary Nanopore Analysis Systems

As will be described in greater detail here, nanopore analysis systems and methods of use thereof, and nanopore devices and methods of fabrication thereof are provided. By way of example, some embodiments provide for nanopore analysis systems having partitioned nanopores configured to receive polymers. The polymers can be sorted by an electrophoretic device in communication with the nanopore device. For example, the electrophoretic device can be a capillary electrophoresis device. The electrophoretic device is in communication, for example fluid communication and/or electrical communication, with a nanopore device and is configured to deliver polymers, for example sorted polymers, to the partitioned nanopores in the nanopore device. Generally, the partitioned nanopores are configured to receive polymers separated in at least one dimension, typically in at least two dimensions or more. The sorted polymers are translocated through a nanopore. The nanopore is configured with a sensing device (e.g., a sensor) for distinguishing or identifying individual monomers of a polymer as the polymer traverses the nanopore. One representative sensing device, among others, includes a resonant tunneling electrode. The resonant tunneling electrode can detect and measure tunneling current as the polymer translocates through the nanopore. The measured tunneling current can be correlated to a predetermined tunneling current indicative of a specific monomer, for example a purine or pyrimidine nucleotide or base.

It should be noted that by increasing the number of times a single species of a polymer is sequenced through the nanopore, inaccuracies in sequencing can be identified and reduced, thereby providing a method of nanopore sequencing with a higher degree of fidelity than presently available.

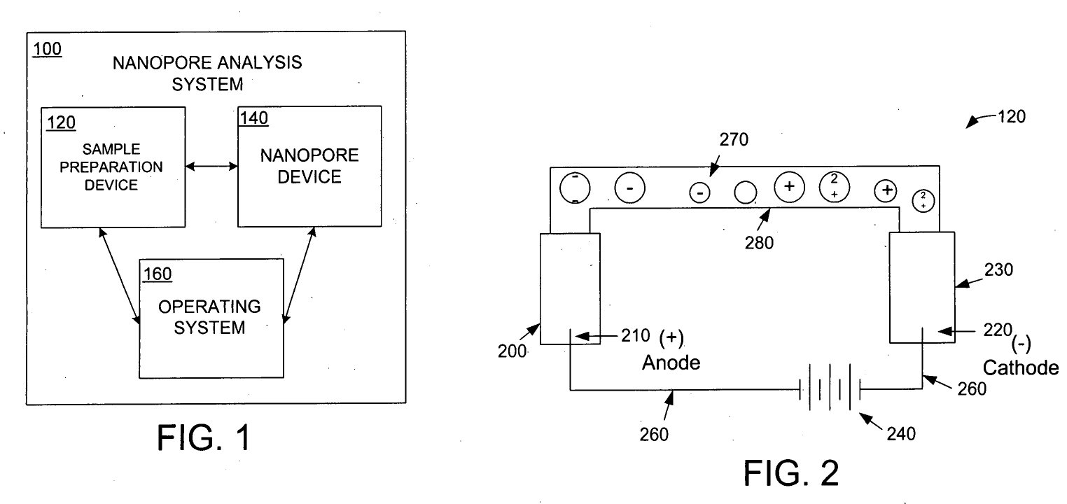

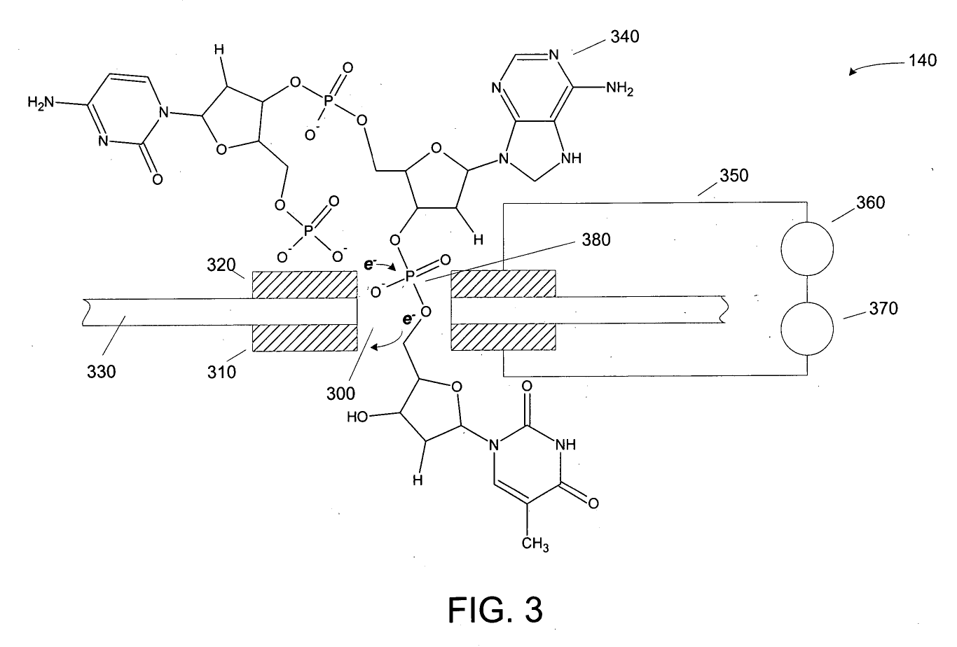

FIG. 1 shows a graphical representation of an exemplary nanopore analysis system 100. Nanopore analysis system 100 comprises a sample preparation device 120 in fluid, and optionally electrical, communication with a nanopore device 140. The nanopore device 140 includes, but is not limited to, a nanopore detection system. FIG. 3 shows an exemplary nanopore device 140 having an exemplary nanopore 300 coupled with electrodes 310, 320 which are in turn are communicatively coupled so that data regarding the polymer, for example a target polynucleotide, can be measured, detected, processed, or stored.

Nanopore analysis system 100 includes, but is not limited to, an operating system 160. The operating system 160 includes, but is not limited to, electronic equipment capable of measuring characteristics of a polymer, for example a polynucleotide, as it interacts with the nanopore 300, a computer system capable of controlling the measurement of the characteristics and storing the corresponding data, control equipment capable of controlling the conditions of the nanopore device and/or components that are included in the nanopore device 140 that are used to perform the measurements as described below. The nanopore system 100 can also be in communication with a distributed computing network such as a LAN, WAN, the World Wide Web, Internet, or intranet.

The nanopore analysis system 100 can measure characteristics such as, but not limited to, the amplitude or duration of individual conductance or electron tunneling current changes across the nanopore. Typically, conductance occurring through a polymer as it traverses the nanopore 300 is detected or quantified. More specifically, electron tunneling conductance measurements are detected for each monomer of a polymer as each monomer traverses the nanopore 300. Such measurements include, but are not limited to, changes in data which can identify the monomers in sequence, as each monomer can have a characteristic conductance change signature. For instance, the volume, shape, purine or pyrimidine base, or charges on each monomer can affect conductance in a characteristic way. Likewise, the size of the entire polynucleotide can be determined by observing the length of time (duration) that monomer-dependent conductance changes occur. Alternatively, the number of nucleotides in a polynucleotide (also a measure of size) can be determined as a function of the number of nucleotide-dependent conductance changes for a given nucleic acid traversing the nanopore. The number of nucleotides may not correspond exactly to the number of conductance changes, because there may be more than one conductance level change as each nucleotide of the nucleic acid passes sequentially through the nanopore. However, there can be a proportional relationship between the two values that can be determined by preparing a standard with a polynucleotide having a known sequence.

Having described an exemplary nanopore system in general, representative components of a representative nanopore system will be described in more detail.

Sample Preparation Device

In one embodiment, the sample preparation device 120 is an electrophoretic device. The electrophoretic device 120 sorts and optionally groups or stacks similar polymers, for example polymers of a specific mass or range of masses, molecular weight, size, charge, conformation (including single or double stranded conformations), or charge-to-mass ratio, to be received by and/or through the partitioned nanopore 300 and detected, for example by a resonant tunneling electrode. Providing multiple polymers of similar or identical characteristics allows for collection of multiple data points for the same polymer or analyte. The multiple data points can be analyzed, for example statistically analyzed, to increase the fidelity of the result. For example, the sequence of monomers in a polymer can be determined. Some data points may incorrectly represent a characteristic of the polymer being analyzed, for example, an incorrect sequence of monomers. Incorrect, or outlying data points can be ignored or deleted from the data set to produce a more reliable and statistically significant result.

Sample Sorting and Stacking

In some embodiments of the disclosed nanopore analysis system, a plurality of polymers may be sorted, stacked, or separated with the electrophoretic device 120 using conventional techniques including, but not limited to, electrophoresis, capillary electrophoresis, molecular sieves, antibody capture, chromatography, affinity chromatography, polynucleotide capture, chromatography, reverse phase chromatography, and ion exchange chromatography.

Capillary electrophoresis (CE) is a family of related techniques that employ narrow-bore (20-200 mm i.d.) capillaries to perform high efficiency separations of both large and small molecules. These separations are facilitated by the use of high voltages, which may generate electroosmotic flow, electrophoretic flow, or a combination thereof, of buffer solutions and ionic species, respectively, within the capillary. The properties of the separation and the ensuing electropherogram have characteristics resembling a cross between traditional polyacrylamide gel electrophoresis (PAGE) and modern high performance liquid chromatography (HPLC). In one embodiment, the electrophoretic device 120 utilizes a high electric field strength, for example, about 500 V/cm or more. One process that drives CE is electroosmosis. Electroosmosis is a consequence of the surface charge on the wall of the capillary. The fused silica capillaries that are typically used for separations have ionizable silanol groups in contact with the buffer contained within the capillary. The pI of fused silica is about 1.5. The degree of ionization can be controlled mainly by the pH of the buffer.

The negatively-charged wall attracts positively-charged ions from the buffer, creating an electrical double layer. When a voltage is applied across the capillary, cations in the diffuse portion of the double layer migrate in the direction of the cathode, carrying water with them. The result is a net flow of buffer solution in the direction of the negative electrode. In untreated fused silica capillaries most solutes migrate towards the negative electrode regardless of charge when the buffer pH is above 7.0.

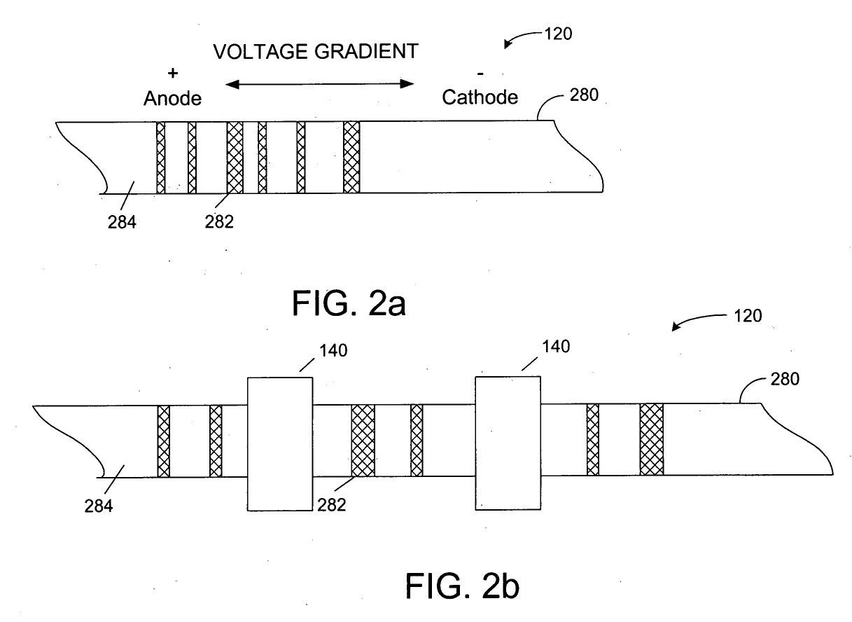

Capillary electrophoresis includes, but is not limited to, capillary zone electrophoresis, isoelectric focusing, capillary gel electrophoresis, isotachophoresis, and micellar electrokinetic capillary chromatography. Capillary zone electrophoresis (CZE), also known as free solution capillary electrophoresis, is the simplest form of CE. The separation mechanism is based on differences in the charge-to-mass ratio. Fundamental to CZE are homogeneity of the buffer solution and constant field strength throughout the length of the capillary. Following injection and application of voltage, the components of a sample mixture separate into discrete zones, as shown in FIG. 2a.

With isoelectric focusing (IEF), a molecule will migrate so long as it is charged, and will stop when it becomes neutral. IEF is run in a pH gradient where the pH is low at the anode and high at the cathode. The pH gradient is generated with a series of zwitterionic chemicals known as carrier ampholytes. When a voltage is applied, the ampholyte mixture separates in the capillary. Ampholytes that are positively charged will migrate towards the cathode while those negatively charged migrate towards the anode. It will be appreciated that the pH of the anodic buffer must be lower than the isoelectric point of the most acidic ampholyte to prevent migration into the analyte. Likewise, the catholyte must have a higher pH than the most basic ampholyte.

Nucleic acids are generally electrophoresed in neutral or basic buffers as anions with their negatively charged phosphate groups. For small DNA fragments, e.g., nucleosides, nucleotides, and small oligonucleotides, free-solution techniques (CZE, MECC) can be applied-generally in conjunction with uncoated capillaries. Alternatively, separation of larger deoxyoligonucleotides is accomplished using capillary gel electrophoresis, generally with coated capillaries, in which, as the name implies, the capillary is filled with an anticonvective medium such as polyacrylamide or agarose. The gel suppress electroosmotic flow and acts a sieve to sort analytes by size. Oligonucleotides, for example poly(dA)40-60 can be separated using this method with a gel of 8% monomer and a buffer consisting of 100 mM Tris-borate, pH 8.3 with 2 mM EDTA and 7 M urea, in under 35 min. with unit base resolution.

Isotachophoresis relies on zero electroosmotic flow, and the buffer system is heterogeneous. This is a free solution technique, and the capillary is filled with a leading electrolyte that has a higher mobility than any of the sample components to be determined. Then the sample is injected. A terminating electrolyte occupies the opposite reservoir, and the ionic mobility of that electrolyte is lower than any of the sample components. Separation will occur in the gap between the leading and terminating electrolytes based on the individual mobilities of the analytes.

Micellar electrokinetic capillary chromatography (MECC) is a free solution technique that uses micelle-forming surfactant solutions and can give rise to separations that resemble reverse-phase liquid chromotography with the benefits of capillary electrophoresis. Unlike isoelectric focusing, or isotachophoresis, MECC relies on a robust and controllable electroosmotic flow. MECC takes advantage of the differential partitioning of analytes into a pseudo-stationary phase consisting of micelles. Ionic, nonionic, and zwitterionic surfactants can be used to generate micelles. Representative surfactants include, but are not limited to, SDS, CTAB, Brij, and sulfobetaine. Micelles have the ability to organize analytes at the molecular level based on hydrophobic and electrostatic interactions. Even neutral molecules can bind to micelles since the hydrophobic core has very strong solubilizing power.

FIG. 2 describes an exemplary sample preparation device 120. The sample preparation device can be an electrophoretic device using a voltage gradient to separate various polymers or analytes. The reservoir 200 receives a sample, for example a sample containing a plurality of polynucleotides. The sample is preferably a fluid sample in a solution buffered to a desired pH and ionic strength. Generally, the electrophoretic device has an anode buffer 210 in the reservoir 200 and a cathode buffer 220 in the reservoir 230. One of skill in the art will appreciate that the pH of the buffer and ionic strength can each be modulated, which in turn can modulate the electrophoretic separation of the polymers or analytes. Additionally, viscosity builders, surfactants, denaturing agents, or other additives can be added to the sample or buffer to vary the separation resolution of the polymers. In some embodiments, capillary electrophoresis requires modifications to the walls of the capillaries, for example capillaries of fused silica. The wall can be modified in a manner to modify or suppress electroosmotic flow, and/or to reduce unfavorable wall-analyte interactions. In one embodiment, the electrophoretic device does not exert electroosmotic flow on the polymers to be separated. For example, a tube 280 of the sample preparation device can have surfaces that are neutral or uncharged during electrophoresis. Charged surfaces of the tube 280 can optionally be coated, for example with an ionic surfactant such as a cationic or anionic surfactant. Exemplary coating substances or buffer additives include, but are not limited to, SDS, cetyltrimethylammonium bromide (CTAB), polyoxyethylene-23-lauryl ether; sulfobetaine (BRIJ), TWEEN, MES, Tris, CHAPS, CHAPSO, methyl cellulose, polyacrylamide, PEG, PVA, methanol, acetonitrile, cyclodextrins, crown ethers, bile salts, urea, borate, diaminopropane, and combinations thereof. Neutralizing charged surfaces of the tube 280 can eliminate or reduce electroosmotic flow. Alternatively, modifications to the surface of the tube 280 or to the buffers can eliminate or reverse the electroosomotic force. For example, neutral deactivation with polyacrylamide eliminates the electroosmotic flow. This results from a decreased effective wall charge and increased viscosity at the wall. Deactivation with cationic groups can reverse the electroosomotic flow, and deactivation with amphoteric molecules allows one to control the direction of the electroosmotic force by altering the pH.

A wide variety of covalent and adsorbed capillary coatings that completely suppress electroosmotic flow are known in the art and are commercially available. Coatings of covalently bound or adsorbed neutral polymers such as linear polyacrylamide are highly stable, resist a variety of analytes, and reduce electroosmotic flow to almost undetectable levels. Such coatings are useful for a wide range of applications including DNA and protein separations, with the requirement that all analytes of interest migrate in the same direction (e. g., have charge of the same sign). For many other applications, however, electroosmotic flow can be used as a pump to mobilize analytes of both positive and negative charge (e.g., a mixture of proteins with a wide range of isoelectric points), or to mobilize species with very low electrophoretic mobility. Bare fused silica capillaries exhibit strong cathodal electroosmotic flow, but are prone to adsorption of analytes, leading to irreproducible migration times and poor peak shapes.

In one embodiment, electrophoretic separation of polymers occurs in the tube or capillary 280. The tube 280 can be a capillary tube and can be coated or uncoated. Exemplary capillary tubes are typically about 0.5 meters or less, more typically about 1 to about 100 cm, and have an interior diameter of about 100 nm or less. The tube 280 can be made of fused silica, glass, quartz, or polymeric substances such as polyurethane, polycarbonate, or polysiloxane.

FIG. 2a is sectional view of the tube 280 showing bands or zones 282 of polymers as they are separated along a voltage gradient. Generally, the samples move from anode to cathode; however, one of skill in the art will recognize that the polarity can be reversed by changing the buffer system, adding ionic surfactants to the sample or buffer, or coating the interior surfaces of the capillary to reduce or eliminate electroosmotic flow. Typically, the polymers in one band 282 are of uniform size, uniform number of monomers, and optionally uniform sequence.

A power source 240 provides a voltage gradient between the anode 210 and the cathode 220. The power source 240 is in electrical communication with reservoirs 200, 230 using conventional electrical conductors 260. Generally, the power source 240 can supply about 10 to about 60 kV, typically about 30 kV. It will be appreciated that the voltage can be adjusted to modify polymer separation. When a voltage gradient is established, individual polymers will move along the voltage gradient, for example according to their charge, mass, or charge-to-mass ratio. In conventional capillary electrophoresis, small positively charged polymers will move quickly from the anode towards the cathode. Larger positively charged polymers will follow with large negatively charged polymers traveling at the end of the sample.

During separation, the polymers travel through the tube 280, for example a capillary tube. The tube 280 can be filled with a separation matrix 270 and a buffer to maintain ionic and pH conditions. The ionic and pH conditions can be optimized to increase the separation resolution of specific polymers. It will be appreciated by one of skill in the art that the different polymers may use different buffers, ionic concentrations, voltages, and separations times to achieve separation of a specific polymer or group of polymers. Representative separation matrices include, but are not limited to, polymers including polyacrylamides, methacrylates, polysiloxanes, agarose, agar, polyethylene glycol, cellulose, or any other substance capable of forming a meshlike framework or sieve. The separation matrix can be colloids, “polymer solutions,” “polymer networks,” “entangled polymer solutions,” “chemical gels,” “physical gels,” and/or “liquid gels.” More particularly, the separation matrix can be a relatively high-viscosity, crosslinked gel that is chemically anchored to the capillary wall (“chemical” gel), and/or a relatively low-viscosity, polymer solution (“physical” gel). The mesh or sieve will work to retain or hinder the movement of large polymers, whereas small polymers will travel quicker through the mesh. Polymers having similar or identical characteristics such as lengths, molecular weights, or charges, will stack together or travel in,bands 282. It will be appreciated that the size of the pores of the separation matrix can be varied to separate different polymers or groups of polymers.

In another embodiment, the reservoir 200 or the tube 280 of the nanopore analysis system can be coated with a substance that specifically binds to a specific polymer or group of polymers. For example, a surface of the reservoir 200 or the tube 280 can be coated with an antibody that specifically binds directly or indirectly to a specific polymer such as a polypeptide or polynucleotide. Alternatively, a polynucleotide having a predetermined sequence can be attached to a surface of the reservoir 200 of the disclosed nanopore analysis system. Suitable polynucleotides are at least about 6 nucleotides, typically about 10 to about 20 nucleotides, even more typically, about 6 to about 15 nucleotides. It will be appreciated that any number of nucleotides can be used, so long as the polynucleotide can specifically hybridize with its complementary sequence or polymers containing its complementary sequence.

Another embodiment provides a nanopore analysis system having polypeptides attached to an interior surface of a reservoir, tube, or capillary. The attached polypeptides can specifically bind to another polypeptide. Exemplary attached polypeptides include, but are not limited to, polyclonal or monoclonal antibodies, fragments of antibodies, polypeptides, for example polypeptides that form dimers with other polypeptides, or polypeptides that specifically associate with other polypeptides to form macromolecular complexes or complexes of more than one polypeptide.

In other embodiments, binding agents are attached to a matrix or resin that is placed inside a reservoir or tube. The binding matrix or resin can be replaced or recharged as needed. The resins or underlying matrices are inert and biologically inactive apart from the binding agent coupled thereto, and can be plastic, metal, polymeric, or other substrate capable of having a binding agent attached thereto. The binding agent can be, but is not limited to, a polypeptide, small organic molecule, nucleic acid, biotin, streptavidin, carbohydrate, antibody, or ionic compound, fragments thereof, or combinations thereof.

In one embodiment, the reservoir 200 receives a plurality of polymers containing a target polymer. Polymers in the sample that are not the target polymer are captured by a binding molecule attached to the surface of the reservoir 200, a tube, or capillary of the nanopore analysis system and are immobilized. The target polymer is mobile and is transported through the nanopore analysis system.

In another embodiment, the target polymer is specifically immobilized by a binding agent such as a polypeptide or polynucleotide. Other polymers are flushed through the nanopore analysis system. Once the other polymers are separated from the target polymer, the target polymer is released from the binding agent, for example by changing pH, ionic strength, temperature, or a combination thereof. Data from the target polymer can then be captured by the nanopore analysis system as the target polymer travels through the nanopore analysis system.

Reactions

Other embodiments provide reservoirs, wells, or modified tubes, of the nanopore analysis system that are configured to perform, facilitate, or contain reactions, for example chemical or enzymatic reactions, on a sample containing a plurality of polymers. In one embodiment, a reservoir or tube can be configured to perform polynucleotide amplification or primer extension using, for example, polymerase chain reaction (PCR).

PCR and methods for performing PCR are known in the art. In order to perform PCR, at least a portion of the sequence of the polynucleotide, for instance a DNA polymer, to be replicated or amplified must be known. Short oligonucleotides (containing about two dozen nucleotides) or primers that are precisely complementary to the known portion of the DNA polymer at the 3′ end are synthesized. The DNA polymer sample is heated to separate its strands and is mixed with the primers. If the primers find their complementary sequences in the DNA, they bind to them. Synthesis begins (as always 5′→3′) using the original strand as the template. The reaction mixture must contain all four deoxynucleotide triphosphates (dATP, dCTP, dGTP, dTTP) and a DNA polymerase, for example a DNA polymerase that is not denatured by the high temperature needed to separate the DNA strands. Suitable heat-stable DNA polymerases are known in the art and include, but are not limited to Taq polymerase.

Polymerization continues until each newly-synthesized strand has proceeded far enough to contain the site recognized by a flanking primer. The process is repeated, with each cycle doubling the number of DNA molecules. Using automated equipment, each cycle of replication can be completed in less than 5 minutes. After 30 cycles, what began as a single molecule of DNA has been amplified into more than a billion copies. It will be appreciated that Reverse Transcriptase PCR is also within the scope of this disclosure.

Other exemplary reactions include fragmenting polymers of a sample. Fragmenting a polymer can be accomplished enzymatically using proteases, peptidases, endonucleases, exonucleases, ribonucleases, physical shearing, sonication, and combinations thereof. Reagents for fragmenting polymers, for example nucleic acids and proteins are known in the art and are commercially available.

Nanopore Device

In one embodiment, as shown in FIG. 2b, the electrophoretic device 120 is coupled to the nanopore device 140 so that the nanopore device 140 receives electrophoretically separated polymers for analysis. In another embodiment a plurality of polymers of the same sequence are delivered to the nanopore device 140 in discrete amounts or bands from the electrophoretic device 120. In still another embodiment, the nanopore device 140 is electrically insulated from the electrophoretic device 120, while optionally remaining in fluid communication with the electrophoretic device 120. Electrically insulating the two components allows for different voltage gradients to be applied in the different components. In another embodiment, the electrophoretic device 120 is in electrical communication with the nanopore device 140 such that the voltage gradient maintained in the electrophoretic device 120 is also maintained in the nanopore device 140. In still another embodiment, the polarity of the electrophoretic device 120 is maintained in the nanopore device 140. In another embodiment, the polarity of the electrophoretic device 120 is different than the polarity of the nanopore device 140.

FIG. 3 shows a diagram of an exemplary nanopore device 140. Generally, the nanopore device 140 comprises a nanopore 300 through which a target polymer 340 traverses, for example in response to a voltage gradient. In one embodiment, the polymer 340 moves from a first side through the nanopore 300 to a second side along a voltage gradient. It will be appreciated that a buffering solution on the first side of the nanopore 300 can be formulated for either the cathode or anode, and the buffer on the second side can be formulated for the corresponding anode or cathode. In one embodiment, the nanopore 300 can be formed in an electrode 320 without an intervening layer 330. Alternatively, the electrodes 310, 320 can be positioned adjacent the nanopore 300 formed in the layer 330. Typically, the nanopore 300 can have a diameter of about 3 to 5 nanometers (e.g., for analysis of single or double stranded polynucleotides), and from about 2 to 4 nanometers (e.g., for analysis of single stranded polynucleotides).

A polymer, for example a polynucleotide, is generally negatively charged. It will be appreciated that a polymer can be reacted with a charge-conferring substance to provide a uniform unit of charge per monomer of the polymer. For example, polymers can be combined with ionic detergents which can provide a net positive or negative charge to the polymer. Nucleic acids are generally negatively charged, and they can be moved through the nanopore device 140 using electroosmotic flow, electrophoresis, or a combination thereof by establishing a voltage gradient between the two sides of the nanopore 300. In one embodiment, the surfaces of the nanopore device 140 can be negatively charged so that positive ions in the sample buffer interact with the negatively charged surface allowing positive ions in the mobile buffer to be drawn to the cathode and subsequently drag other solutes, for example negatively charged polymers, in the sample solution with them.

Generally, a power supply 370 maintains a voltage gradient or voltage differential between the two sides on either side of the nanopore 300 such that a polymer, for example a net negatively charged polymer, will travel down the voltage gradient and though the nanopore 300. In one embodiment, the nanopore 300 is generally about 100 nm or less in diameter at its widest point. It will be appreciated that the size of the aperture can vary from about 1 nm to about 100 nm, typically about 2.5 nm to 5 nm, depending on the type of polymer to be analyzed. In one embodiment, the nanopore 300 is of a diameter or width sufficient to permit one monomer of one target polymer to traverse the aperture at a time.

The nanopore 300 is typically formed in an insoluble substrate 330 which separates two compartments. The substrate 330 generally is formed of a non-conductive substance including but not limited to silicates, aluminosilicates, glass, quartz, silicon, nitride, silicon oxide, mica, polyimide, carbon based materials, thermoplastics, elastomers, polymeric materials, Si₃N₄ and the like. Methods of manufacturing nanopores are known in the art and include, but not limited to, spontaneous assembly of molecules such as lipids and proteins, etching such as ion etching, optical lithography, and electron-beam lithography, to name a few. The substrate 330 can have a single nanopore 300 or a plurality of nanopores 330 as depicted in FIG. 3.

The nanopore device 140 can be fabricated using various techniques and materials. The nanopore 300 can be made in a thin (500 nm) freestanding silicon nitride (SiN₃) membrane supported on a silicon frame. Using a Focused Ion Beam (FIB) machine, a single initial pore of roughly 500 nm diameter can be created in the membrane. Then, illumination of the pore region with a beam of 3 KeV Argon ions sputters material and slowly closes the hole to the desired dimension of roughly 2 nm in diameter (See Li et al., “Ion beam sculpting at nanometer length scales”, Nature, 412: 166-169, 2001, which is incorporated herein by reference). Metal electrodes are formed by evaporation or other deposition means on the opposing surfaces of the SiN₃ membrane. Wire bonding to the metal electrodes allows connection to the tunneling current bias and detection system. The bias is applied using an AC source with the modest requirement of roughly 3-5 volts at 30-50 MHz. The tunneling currents are expected to be in the nanoamp range, and can be measured using a commercially available patch-clamp amplifier and head-stage (Axopatch 200B and CV203BU, Axon Instruments, Foster City, Calif.).

As noted, the nanopore device 140 also includes a detector 310, such as an electrode or other sensing device, for collecting data from the polymer as it traverses the nanopore 300. The detector can be configured to surround the edge of the nanopore 300, and optionally can include more than one detector. The detectors can be configured to detect or collect different types of data as the polymer traverses the nanopore 300, including but not limited to, conductivity, ionic current, tunneling current, temperature, resistance, impedance, fluorescence, radioactivity, or a combination thereof. The data collected, recorded, or transmitted by the detectors can be correlated to specific monomers as the polymer traverses the nanopore 300 such that the sequence of monomers forming the polymer can be ascertained. For example, the data obtained from monomers of a specific polymer can be correlated to predetermined values indicative of a specific monomer. The predetermined values can be calculated or determined from polymers of a known sequence of monomers. A gauge 360 can be in communication with the detector 310 using wires or conductors 350, and can display data or changes in data such as voltage or current as individual monomers or polymers travel through the nanopore 300.

Partitioned Nanopore

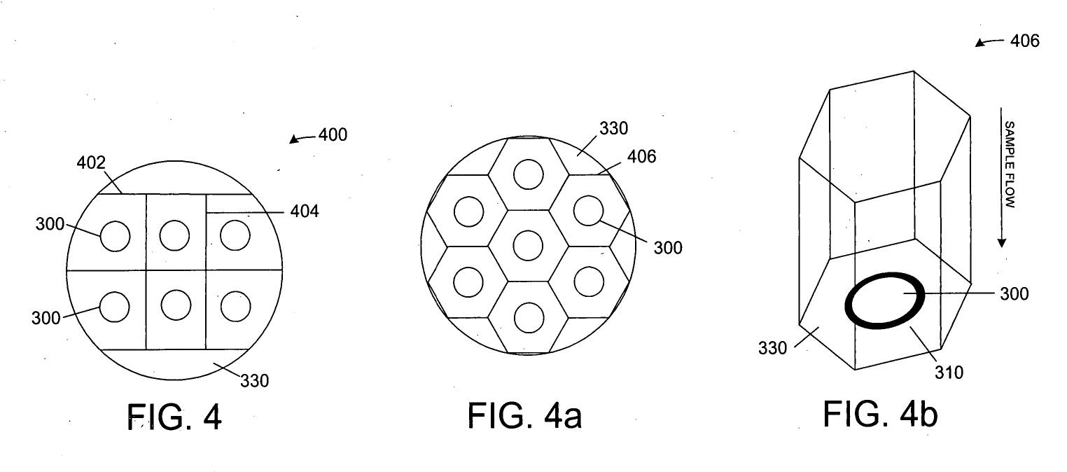

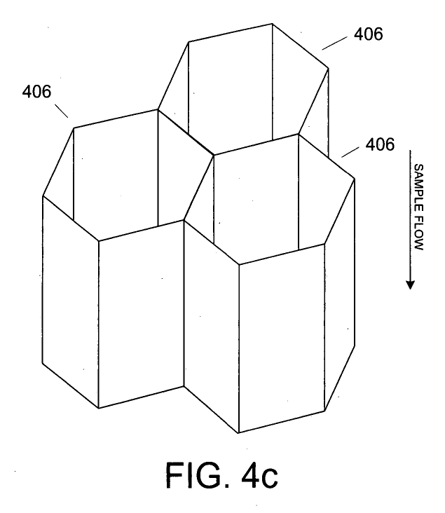

FIGS. 4 and 4a-c show an alternative embodiment in which substrate 330 has a plurality of nanopores 300 disposed thereon. Each nanopore 300 is partitioned or segregated from the other nanopores such that different fractions of a polymer sample are directed to each nanopore 300. Generally, the polymer sample is processed by the sample preparation device 120, for example sorted according charge-to-mass ratio. This first processing of the polymer sample can be performed along a first axis, for example along a vertical axis. The sample can be further separated along a second axis, for example along a horizontal axis. The second separation can be accomplished by changing the direction of the applied electric field from vertical to horizontal. Alternatively, the sample can separated along the second axis by pH, isolelectric point, mass, charge, and/or binding affinity for a target compound.

Accordingly, one embodiment provides separating a polymer sample in at least two dimensions. In some embodiments, the separation in each dimension will use the same or different separation techniques. FIG. 4 shows one embodiment having the substrate 330 configured with the nanopore 300 partitioned with horizontal barriers 402 and vertical barriers 404. The partitioning barriers can be in any geometric shape, including, but not limited to, linear, circular, square, elliptical, rectangular, oviod, or polygonal including, but not limited to hexagonal. In one embodiment, the partitioning barriers form a conical structure around the nanopore. The conical structure has a wide opening at a first end for receiving polymers from the sample preparation device 120. The conical structure narrows towards the nanopore to funnel and or align polymers with the nanopore as the polymers travel through the conical structure of the partitioned nanopore.

FIG. 4a illustrates one embodiment in which hexagonal partitioned nanopores 406 are disposed in substrate 330. In this embodiment, partitioned nanopores 406 are uniformly placed in substrate 330.

FIG. 4b is a perspective view of a representative partitioned nanopore 406. The barriers forming the partition can extend perpendicularly from substrate 330 for a distance sufficient to prevent crossover of separated polymers from one partitioned nanopore 406 to an adjacent partitioned nanopore 406. The partitioning barrier can be made of a durable and impermeable material such as silicon, metal, metal alloys, aluminum, ceramic, or an impermeable polymer. Generally, the barriers extend from about 1 μm to about −10 mm, typically from about 5 μm to about 1 mm, more typically from about 10 μm to about 50 μm. In other embodiments, the barriers extend from the substrate 330 to the interface of sample preparation device 120. In still other embodiments, the barriers can extend into the separation matrix of sample preparation device 140.

FIG. 4c is a diagram of an exemplary partitioning grid. The grid can be fitted to cover a plurality of nanopores 300. It will be appreciated that some openings of the grid can be sealed prior to use. The closed openings in the grid generally correlate to positions having no nanopore on the substrate 330. Alternatively, certain nanopores can be bypassed by using a grid having closed openings corresponding to the nanopores to be bypassed. The grid can be removably inserted or fitted onto the nanopore device 140. The grid can also be covered with a mesh or screen to prevent large aggregates of polymers from passing through and blocking a nanopore. The size of the openings of the mesh or screen can vary depending on the nature and characteristics of the polymers being analyzed. Generally, the opening or pores of the mesh or screen will be about 200 nm in diameter or approximately twice the diameter of the nanopore.

One embodiment provides partitioned nanopores in predetermined positions for detecting polymers separated in at least two dimensions. It will be appreciated that a target polymer can have a specific separation profile, depending on the number and variety of separation techniques used on a polymer sample. For example, a test sample may contain two or more target polymers. The separation techniques can be chosen such that a first target polymer traverses a first partitioned nanopore at a first predetermined position and a second target polymer traverse a second partitioned nanopore at a second predetermined position. A detectable signal from the first partitioned nanopore indicates that the test sample contains the first target polymer. A detectable signal from the second partitioned nanopore indicates that the test sample contains the second target polymer. In one embodiment, a detectable signal from each partitioned nanopore is correlated to the presence of a specific polymer, type of polymer, class of polymers, or polymers having a specific sequence of monomers. The correlation can be based on separation profiles of the polymers in at least two dimensions. Suitable two dimensional separation techniques are known in the art.

Detectors

As noted above, nanopore analysis system 100 includes at least one detector for collecting data as a polymer interacts with the nanopore 300. The data can be used to determine the sequence of monomers forming the polymer. The data can be electromagnetic, conductive, colorometric, fluorometric, radioactive response, or a change in the velocity of electromagnetic, conductive, colorometric, fluorometric or radioactive component. Detectors can detect a labeled compound, with typical labels including fluorographic, colorometric, and radioactive components. Example detectors include resonant tunneling electrodes, spectrophotometers, photodiodes, microscopes, scintillation counters, cameras, film and the like, as well as combinations thereof. Examples of suitable detectors are widely available from a variety of commercial sources known to persons of skill in the art.

In one embodiment, the detection system is an optical detection system and detects for example, fluorescence-based signals. The detector may include a device that can expose a polymer to an exciting amount of electromagnetic radiation in an amount and duration sufficient to cause a fluorophore to emit electromagnetic radiation. Fluorescence is then detected using an appropriate detector element, e.g., a photomultiplier tube (PMT). Similarly, for screens employing colorometric signals, spectrophotometric detection systems are employed which detect a light source at the sample and provide a measurement of absorbance or transmissivity of the sample.

Other embodiments provide a detection system having non-optical detectors or sensors for detecting particular characteristic(s) or physical parameter(s) of the system or polymer. Such sensors optionally include temperature (e.g., when a reaction produces or absorbs heat, or when the reaction involves cycles of heat as in PCR or LCR), conductivity, potentiometric (pH, ions), and/or amperometric (for compounds that can be oxidized or reduced, e.g., O₂, H₂O₂, I₂, oxidizable/reducible organic compounds, and the like) sensors/detectors.

Still other detectors are capable of detecting a signal that reflects the interaction of a receptor with its ligand. For example, pH indicators, which indicate pH effects of receptor-ligand binding, can be incorporated into the device along with the biochemical system (e.g., in the form of an encapsulated cell) whereby slight pH changes resulting from binding can be detected. Additionally, the detector can detect the activation of enzymes resulting from receptor ligand binding, e.g., activation of kinases, or detect conformational changes in such enzymes upon activation, e.g., through incorporation of a fluorophore that is activated or quenched by the conformational change to the enzyme upon activation. Such reporter molecules include, but are not limited to, molecular beacons.

Resonant Tunneling Electrode

Another embodiment provides a nanopore analysis system comprising a resonant tunneling electrode. Resonant tunneling electrodes and methods of their use in sequencing polymers are disclosed in U.S. Patent Application Publication Nos. 20040149580 and 20040144658 to Flory, both of which are incorporated by reference in their entireties.

The electrodes 310 and 320 shown in FIG. 3 form a representative resonant tunneling electrode configured to obtain data from polymers interacting with the nanopore 300. The term “resonant” or “resonant tunneling” refers to an effect where the relative energy levels between the current carriers in the electrodes are relatively similar to the energy levels of the proximal polymer segment. This provides for increased conductivity. Resonant tunneling electrodes measure or detect tunneling current, for example from one electrode 320 through a biopolymer 340 to another electrode 310.

The electrodes 310, 320 can be formed in whole or part of one or more of a variety of electrically conductive materials including but not limited to, electrically conductive metals and alloys. Exemplary metals and alloys include, but are not limited to, tin, copper, zinc, iron, magnesium, cobalt, nickel, silver, platinum, gold, and/or vanadium. Other materials well known in the art that provide for electrical conduction may also be employed. When the electrode 320 is deposited on or comprises a portion of the solid substrate 330, it may be positioned in any location relative to the second electrode 310. Electrodes 310, 320 are typically positioned in such a manner that a potential can be established between them. In operation, biopolymer 340 is generally positioned sufficiently close to electrodes 310, 320 so specific monomers and their sequence in biopolymer 340 can be detected and identified. It will be appreciated that the resonant tunneling electrode can be fitted to the shape and configuration of the nanopore 300. Accordingly, electrodes 310, 320 that may be used with nanopore 300 can be curved parts of rings or other shapes. The electrodes can also be designed in broken format or spaced from each other. However, the design should be capable of establishing a potential across electrode 320, and the nanopore 300 to the electrode 310.

Exemplary Methods of Use

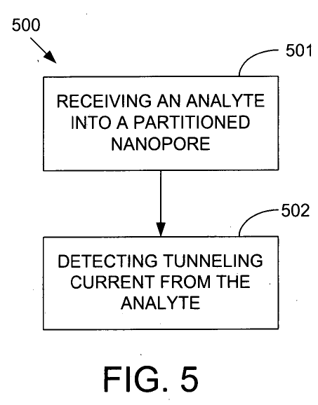

FIG. 5 shows an exemplary method for characterizing an analyte according to the present disclosure. The process 500 begins by receiving an analyte into a partitioned nanopore, as described in step 501. In step 502, tunneling current is detected from the analyte using a set of resonant tunneling electrodes.

Another embodiment provides a method in which more than one target analyte is characterized. In this method, a group of analytes are separated according to a physical characteristic of the analytes or more than one physical characteristic of the analytes. For example, the analytes can be electrophoretically separated and optionally separated based on binding affinity to a substrate. The analytes can be separated into groups of analytes having the same characteristics, including, but not limited to the same or approximately the same sequence of monomers. Analytes of a first group can be received into a predetermined partitioned nanopore, and the analytes of a second group having at least one characteristic different than the first group can be received into a second partitioned nanopore. The characteristics of the different analytes can be analyzed, for example by detecting resonant tunneling current as the analytes traverse their respective nanopores. If the analytes are, for example, polynucleotides, the sequence of the two groups of analytes can be determined. Thus, the present disclosure encompasses multiplexing or simultaneously determining the sequence of at least two target polymers, for example biopolymers.

Another embodiment provides a method for obtaining the sequence of a polymer, for example a biopolymer such as a polypeptide or polynucleotide using the disclosed nanopore analysis system 100. Nanopore sequencing of polynucleotides has been described (U.S. Pat. No. 5,795,782 to Church et al.; U.S. Pat. No. 6,015,714 to Baldarelli et al., the teachings of which are both incorporated herein by reference in their entireties). In general, nanopore sequencing involves detecting monomers of a polymer as the polymer moves down a voltage gradient established between two regions separated by the nanopore 300. The nanopore 300 between the regions is capable of interacting sequentially with the individual monomer residues of a polynucleotide present in one of the regions. Nanopore-dependent measurements are continued over time, as individual monomer residues of the polynucleotide interact sequentially with the interface, yielding data suitable to infer a monomer-dependent characteristic of the polynucleotide. In some embodiments, the monomer-dependent characterization achieved by nanopore sequencing of the disclosed nanopore analysis system 100 may include identifying physical characteristics such as, but not limited to, the number and composition of monomers that make up each individual polynucleotide, in sequential order.

The term “sequencing” as used herein means determining the sequential order of monomers in a polymer, for example nucleotides in a polynucleotide molecule. Sequencing as used herein includes in the scope of its definition, determining the nucleotide sequence of a polynucleotide in a de novo manner in which the sequence was previously unknown. Sequencing as used herein also includes in the scope of its definition determining the nucleotide sequence of a polynucleotide wherein the sequence was previously known. Sequencing polynucleotides, the sequences of which were previously known, may be used to identify a polynucleotide, to confirm a polynucleotide, or to search for polymorphisms and genetic mutations.

Biopolymers sequenced by nanopore analysis system 100 can include polynucleotides comprising a plurality of nucleotide monomers, for example nucleotide triphosphates (NTPs). The nucleotide triphosphates can include naturally occurring and synthetic nucleotide triphosphates. The nucleotide triphosphates can include, but are not limited to, ATP, dATP, CTP, dCTP, GTP, dGTP, UTP, TTP, dTTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Preferably, the nucleotide triphosphates are selected from the group consisting of dATP, dCTP, dGTP, dTTP, dUTP, and combinations thereof. Modified bases can also be used instead of or in addition to nucleotide triphosphates and can include, but are not limited to, 5-Br-UTP, 5-Br-dUTP, 5-F-UTP, 5-F-dUTP, 5-propynyl dCTP, and 5-propynyl-dUTP. Additionally, the nucleotides can be labeled with a detectable label, for example a label that modulates resonant tunneling current including, but not limited to, metal particles of about 100 nm in diameter or less.

Detection of Mutations

Another embodiment provides a method for detecting a variant of a first nucleic acid. A variant of a polymer generally has a different sequence than the corresponding polymer, typically a difference of less than 5 monomers, more typically a difference of 1 monomer. A variant of a nucleic acid includes, but is not limited to, single nucleotide polymorphisms, deletions, substitutions, inversions, and transpositions. In operation, a sample comprising a target nucleic acid is amplified, for example using PCR or RT-PCR. Primers and nucleotide mixtures are selected to produce primer extension products such that the length of the primer extension products of a target nucleic acid and a variant of the target nucleic acid differ by at least one nucleotide. For example, if a target nucleic acid has a first nucleotide in a first position, and a variant of the target nucleotide has a second nucleotide in the first position, primers can be selected that bind immediately 3′ of the first position of either the variant or the target nucleotide. A nucleotide mixture for primer extension can be formulated to contain a ddNTP or other chain terminating nucleotide complementary to the second nucleotide in the first position of the variant. Accordingly, if the sample contains the variant, the primer will be extended by one nucleotide, namely the ddNTP. If the sample contains the target nucleotide, it will be extended by at least two nucleotides because the ddNTP in the nucleotide reaction mixture will not be incorporated into the first nucleotide added to the primer extension product. Thus, a variant and target nucleic acid can be distinguished based on size. It will be appreciated that at least one of the nucleotides can be labeled with a detectable label, for example, a fluorophore, or a conductivity modulating agent including, but not limited to, metal particles less than about 100 nm in diameter.

Once the primer extension reaction has been performed, the sample is delivered to the electrophoretic device 280. As the polynucleotide translocates through or passes sufficiently close to the nanopore 300, measurements (e.g., ionic flow measurements, including measuring duration or amplitude of ionic flow blockage, and tunneling current measurements) can be taken by the nanopore detection system 140 as each of the nucleotide monomers of the polynucleotide passes through or sufficiently close to the nanopore 300. The measurements can be used to identify the sequence and/or length of the polynucleotide. Nanopore 300 can be dimensioned so that only a single stranded polynucleotide can translocate through the nanopore 300 at a time or so that a double or single stranded polynucletide can translocate through the nanopore 300.

It should be emphasized that many variations and modifications may be made to the above-described embodiments. All such modifications and variations are intended to be included herein within the scope of this disclosure and protected by the following claims.

What is claimed is:

1. A nanopore device comprising:
a substrate comprising a plurality of partitioned nanopores configured to receive a polymer sample; and
a plurality of sets of resonant tunneling electrodes adjacent the partitioned nanopore, at least one set of resonant tunneling electrodes configured to detect tunneling current as monomers of a polymer in the polymer sample sequentially travel through at least one partitioned nanopore.

2. The nanopore device of claim 1, wherein each of the resonant tunneling electrodes is independently coupled to at least one of the partitioned nanopores.

3. The nanopore device of claim 1, wherein each of the partitioned nanopores is positioned in a predetermined location in the substrate for detecting a predetermined polymer.

4. The nanopore device of claim 1, further comprising:
a power source for supplying an electric field to the nanopore device, wherein the electric field applies a force on the polymer sample that causes the polymer sample to sort into separate fractions, and wherein the electric field draws polymers of each separate fraction through one of the partitioned nanopores.

5. The nanopore device of claim 4, wherein each of the partitioned nanopores is configured to receive a unique fraction of the sorted polymer sample.

6. The nanopore device of claim 4, wherein at least two of the plurality of partitioned nanopores is configured to receive polymer fractions having different sequences of monomers.

7. The nanopore device of claim 1, wherein the device is configured to sequence polymers in the polymer samples, wherein the polymers are separated in at least two dimensions.

8. The nanopore device of claim 1, further comprising at least one sample preparation device in fluid communication with the nanopore detection device.

9. The nanopore device of claim 8, wherein the sample preparation device comprises an electrophoretic device configured to electrophoretically separate the polymer sample into fractions and deliver the fractions to the plurality of partitioned nanopores.

10. The nanopore device of claim 9, wherein the electrophoretic device is a capillary electrophoresis device.

11. The nanopore device of claim 1, wherein the nanopores have a diameter of about 3 to 5 nanometers.

12. A method for characterizing an analyte comprising:
receiving the analyte through a partitioned nanopore; and
detecting tunneling current from the analyte with a set of resonant tunneling electrodes disposed adjacent the partitioned nanopore.

13. A method for sequencing a polynucleotide, the method comprising:
receiving an amplified polynucleotide sample into a capillary operably coupled to a plurality of partitioned nanopores positioned in predetermined locations;
providing an electric field across the capillary to electrophoretically separate the amplified polynucleotide sample into fractions, wherein each fraction comprises at least two polynucleotides having about the same number of monomers;
determining the sequence of each of the two polynucleotides in at least one fraction by detecting tunneling current through the two polynucleotides with a resonant tunneling electrode as the two polynucleotides individually travel through at least one partitioned nanopore in fluid communication with the capillary; and
determining a statistically significant sequence of the amplified polynucleotide based on the detected tunneling currents by correlating the detected tunneling currents to predetermined tunneling currents indicative of specific monomers.

14. The method of claim 13, wherein interior surfaces of the capillary are coated in a manner that reduces or eliminates electroosmotic flow.

15. A nanopore analysis system for determining the sequence of a target polynucleotide, the system comprising:
a plurality of capillary electrophoresis devices, each of the plurality of capillary electrophoresis devices independently and operatively coupled to a partitioned nanopore; and
a resonant tunneling electrode independently and operatively coupled to the partitioned nanopore, wherein the resonant tunneling electrode is configured to detect tunneling current through a polymer as monomers of the polymer sequentially travel through the partitioned nanopore.

16. A nanopore device comprising:
a plurality of nanopores disposed on a substrate for receiving fractions of a polymer sample;
a partitioning grid operatively coupled to the plurality of nanopores for segregating each of the plurality of nanopores; and
a plurality of resonant tunneling electrodes configured to detect tunneling current as monomers of a polymer in the polymer sample sequentially travel through each of the plurality of partitioned nanopores.

17. The nanopore device of claim 16, wherein each of the plurality of resonant tunneling electrodes is independently coupled to one of the plurality of partitioned nanopores.

18. The nanopore device of claim 17, further comprising:
a power source for supplying an electric field to the nanopore device, wherein the electric field applies a force on the polymer sample which causes the polymer sample to sort into separate fractions, and wherein the electric field draws polymers of each separate fraction through one of the partitioned nanopores.

19. The nanopore device of claim 18, wherein each of the plurality of partitioned nanopores receives a unique fraction of the sorted polymer sample.

20. A method for simultaneously determining the sequence of more than one target polynucleotide comprising:
separating a mixture of polynucleotides having different nucleic acid sequences into separate groups, wherein each group comprises polynucleotides of the same sequence;
simultaneously receiving each group of polynucleotides into a separate partitioned nanopore;
simultaneously detecting tunneling current from the each polynucleotide within each separate group with a set of resonant tunneling electrodes disposed adjacent each partitioned nanopore; and
determining a statistically significant sequence of each group of polynucleotides based on the detected tunneling currents.
